(19) United States
(12) Patent Application Publication
(10) Pub. No.: US 2025/0025846 A1
(43) Pub. Date: Jan. 23, 2025

(54) METHODS, DEVICES, SYSTEMS AND KITS FOR PREPARING COMPOSITIONS FOR CARE AND REPAIR OF VARICOSE VEINS

(71) Applicant: VASCULAR BARCELONA DEVICES, S.L., Barcelona (ES)

(72) Inventors: Enrique ROCHE REBOLLO, Barcelona (ES); Jean-Baptiste GALY, Sabadell (ES); Guiu LLUSÀ MELÉNDEZ, Barcelona (ES); Federico GREGO MAYOR, Barcelona (ES)

(21) Appl. No.: 18/910,643

(22) Filed: Oct. 9, 2024

Related U.S. Application Data
(62) Division of application No. 17/180,050, filed on Feb. 19, 2021.
Application No. 17/180,050 is a continuation of application No. PCT/EP2019/072242, filed on Aug. 20, 2019.

(30) Foreign Application Priority Data
Aug. 21, 2018 (EP) 18382621.3

(51) Int. Cl. B01F 23/235; A61J 1/20; A61M 5/178; B01F 33/501; B01F 35/00; B01F 35/71; B01F 35/75; B01F 101/00

(52) U.S. Cl. CPC B01F 23/2351; A61J 1/2096; A61M 5/1782; B01F 33/5014; B01F 35/189; B01F 35/7174; B01F 35/75425; B01F 2101/2202; B01F 2215/0481

(57) ABSTRACT

Methods for the production of foamed sclerosant compositions are provided. Containers include a container body including one or more sidewalls extending between a top and a bottom of the container body and a bottom surface, a foaming space being defined in an interior of the container body. The foaming space contains a sterile gas and a mixing element configured to be operatively coupled with a rotatable actuator without the actuator entering into the foaming space. The container includes a female coupling member for mating with a syringe, and a pressure equalizer for equalizing a pressure inside the foaming space with a pressure outside the foaming space. Systems and kits including such containers are also provided. Also disclosed are methods for the preparation of a sclerosant foam which may include a pressure release before extraction of the foam and/or a continued rotation of the actuator while the foam is being extracted.

### CROSS-REFERENCE TO RELATED APPLICATIONS

This application relates to and claims the benefit and priority to U.S. application Ser. No. 17/180,050, filed Feb. 19, 2021, which relates to and claims the benefit and priority to International Application No. PCT/EP2019/072242, filed Aug. 20, 2019, which relates to and claims the benefit and priority to European Patent Application No. EP18382621.3, filed Aug. 21, 2018.

### FIELD

The present disclosure is related to the technical field of vascular medicine, and more particularly to the field of treatments for varicose veins and other vascular problems such as e.g. spider veins and/or hemorrhoids.

Specifically, the present disclosure relates to devices, systems, kits and methods for obtaining a foam, which can be used for the treatment of the affected veins. The present disclosure further relates to the composition of the foam itself obtained by any of the procedures described throughout the present disclosure, as well as the use of the devices, systems and kits in order to treat varicose veins and other vascular problems such as e.g. spider veins and/or hemorrhoids.

### BACKGROUND

Varicose veins occur when the venous valves (which prevent the backflow of blood) do not work properly. As a result, the vein walls are weakened, and they can become deformed and dilated. Due to the fact that the valves do not work properly, the blood may recirculate and short-circuits may be created. Subsequently, the veins may become progressively dilated. As a result, the varicose veins can become more visible, and can be full of bends and become more voluminous. The evolution of this pathology may lead to consequences beyond the mere cosmetic ones, such as discoloration of the skin, pain and swelling of the extremities due to the effect of the venous hypertension.

According to the Spanish Society of Angiology and Vascular Surgery (SEACV) in Spain, varicose veins affect from 30 to 33% of the adult population in industrialized countries.

The veins most commonly affected are those located in the legs, although varicose veins may occur interiorly as well: e.g. varicose veins in the esophagus, around organs located in the pelvis (pelvic and ovarian varicose veins) or at or near the most distal part of the digestive tube, near the anus (hemorrhoids).

Nowadays, there are many different treatments and/or strategies in order to mitigate or eliminate these problems. Among them, we can find surgical methods. These surgical methods are related to the surgical extraction of the affected veins using classical surgery. This technique has been employed for over 100 years and is based on making different skin incisions with subsequent clamping, ligation and stripping of the venous segments affected. This technique is performed under spinal anesthesia (infiltration of the spinal space) in order to obtain the anesthesia of both limbs, as well as with local or nerve block anesthesia to anesthetize more limited areas.

In the late 1990's, there was an important advance regarding the treatment of this disease because the varicose veins were treated with less invasive techniques such as the endovenous techniques. Among these new methods, it is important to highlight intravenous procedures that apply heat through a catheter (endolaser or radio frequency systems).

These systems were able to reduce injuries due to the fact that the methods are usually performed under ultrasound guidance. The effect to the veins was produced by the release of heat or electricity from inside the vein, thus an internal injury into the vein may be produced. This way, a thrombosis of the veins was achieved.

Another technique is sclerotherapy by injection of a sclerosing foam. This technique (compared to the “Endolaser” or radiofrequency therapies) is even less aggressive, less painful, and needs no anesthesia (R Van den Bos et al. Endovenous therapies varicosites of lower extremity. A meta-analysis. J Vasc Surg 2009 January; 49 (1): 230-9).

In summary, in the late 1990′s and early 2000′s a trend was found to minimize the aggressiveness of varicose veins treatments. Of all the new emerged techniques, sclerotherapy seems to be the less invasive and the one which can be applied to practically any type of varicose vein.

For this reason, in recent years, this procedure has been developed a lot further and there has been a growing interest from the scientific community in order to obtain methods suitable for the manufacture of a foam in a safe and convenient way.

Sclerotherapy involves the injection of a liquid with the ability to irritate the vascular endothelium (a thin layer or lining inside the vein that is in contact with the bloodstream).

This drug or liquid medicine can become a foam when shaken. The products internationally approved for this use are lauromacrogol (also known named as polidocanol and commercially available as etoxiesclerol®), and sodium tetradecyl sulfate.

The advantage of using such a product as a foam is based on the enhancement of its effect due to the larger contact surface with the endothelial wall. The larger contact surface offers the possibility of dose reduction. Also the visibility of the drug as a foam using ultrasounds through the ultrasound scanner is improved (Schadeck M, Allaert FA. Duplex scanning in the mechanism of the sclerotherapy: Importance of the spasm. Phlebology 1995;Suppl1: 574-576).

The effect produced by the foam on the endothelium involves the injury of the cell layer of the vein, whereby thrombosis of its content is produced. Later, this vein suffers a fibrosis process (retraction and disposal) and the vein eventually may disappear after several months.

This process can be faster or slower depending on the size of the vein or the potency of the varicose agent. Therefore, it is sometimes necessary to apply several sessions on the vein.

Although there are several methods in order to eliminate or remove varicose veins, the less aggressive and less disabling treatment so far, and the one which can be used for a wide variety of pathologies is the ultrasound-guided Foam Sclerotherapy using polidocanol or other sclerosing agents. Sclerotherapy is the least invasive treatment of varicose veins known today as it can be performed in a physician's office and in a completely ambulatory way. Therefore, the present disclosure is focused on the use of this technique.

In 1995, Dr. Juan Cabrera presented the results of the application of a foam that he had developed with his son, the pharmacist Juan Cabrera (Cabrera J. et al. “Treatment of Varicose Long Saphenous Veins of Sclerosants in Microfoam Form With: Long-term Outcomes”. Phlebology (2000) 15; 19-23). This foam was characterized by its density and high solubility thanks to the use of a mixture of physiological gases.

Furthermore, he managed to achieve a type of foam with very small and uniform bubble size, thanks to his method of using a mixer. By using a mixture of physiological gases, the foam had greater security and stability. This foam was called “microfoam” due to the small bubble size, uniformity and stability.

Shortly after, the maximum popularization of the sclerosing foam came from Lorenzo Tessari (Tessari L., Cavezzi A., Frullini A., Preliminary experience with a new sclerosing foam in the treatment of varicose veins. Dermatol Surg 2001 January; 27 (1): 58-60) who published his experience using a foam easily manufactured through a procedure called “The Tessari Method”. The method consists in the agitation of a sclerosing liquid using two syringes connected via a three-way tap. By means of successive alternately movements with each of the syringes connected to the gas/liquid mixture, a mix of foam, located at the inner part of the syringe, was achieved. However, this manufacturing technique, in spite of being the most widespread is not the most effective, since a relatively unstable and heterogeneous foam is obtained.

In recent years numerous articles and papers have been published about safety profiles, side effects and potential complications arising from the use of these products. Thus, it appears demonstrated that the best foam used is the one whose gas is a mixture of O2/CO2 at different concentrations. With this arrangement, the solubility and diffusion in blood is very high as opposed to atmospheric gas foams. Additionally, the foam stability is linked to the size of the bubble. Also, it has been found that the foam is more stable when the bubble diameter is more homogeneous.

As has already been mentioned, although there are a lot of manufacturing systems of sclerosing foam, the most common foam (and the one which is normally used around the world) is the foam obtained with the Tessari method. However, this method has many problems of standardization and homogenization. This system consists of mixing air flow with the selected liquid, either polidocanol or sodium tetradecyl sulfate (commercially known as sotradecol®). This foam can have medium size bubbles, but of irregular size and the foam becomes unstable after a few seconds of its formation. Additionally, the use of atmospheric gas as a vehicle for the sclerosing foam limits the foam administrable per session.

U.S. Publication No. 2017/0144115 discloses a container for the production of a foamed sclerosant composition, kits and systems including such a container, and methods for preparing a foamed sclerosant composition using such containers. In an aspect, the container comprises a container body and a mixing element disposed in the container body, such that a foaming space is defined in an interior of the container body between the sidewalls and the mixing element. The mixing element may be configured to be operatively coupled with a rotating actuator without the actuator reaching the foaming space. In particular, the mixing element may have a magnetic core, such that the mixing element can be set in motion when placed on a magnetic stirrer.

International Publication No. WO2017/085209 relates to a container for the production of a foamed sclerosant composition, to kits and systems including such a container, to methods for preparing a foamed sclerosant composition using such containers, and to foamed sclerosant compositions obtainable by such methods. In an aspect, the container comprises a sealed sterile container body having one or more sidewalls extending between a top and a bottom of the container body and defining a foaming space. The container further comprises a mixing element disposed in the container body. The container contains a previously introduced gas and liquid sclerosant composition. The mixing element is configured to be operatively coupled with a rotatable actuator without the actuator reaching the foaming space. A medical professional may select the appropriate quantity and concentration for every treatment.

These documents disclose methods and devices that facilitate quick production of good quality sclerosant foam with relatively simple tools and in a highly sterile manner.

However, there is still a need for systems and methods that further improve the production of sclerosant foam.

### SUMMARY

In a first aspect, the present disclosure provides a container for the production of a foamed sclerosant composition, the container comprising a sealed sterile container body for foaming comprising one or more sidewalls extending between a top and a bottom of the container body, a foaming space being defined in an interior of the container body. The foaming space contains a substantially sterile gas and a mixing element configured to be operatively coupled with a rotatable actuator without the actuator entering into the foaming space. The container comprises a female coupling member for mating with a syringe, and a pressure equalizer for equalizing a pressure inside the foaming space and outside the foaming space.

In accordance with this aspect, a container is provided which is specifically suited for the substantially sterile preparation of a sclerosant foam in which a syringe may be used for the introduction of a liquid sclerosant composition. The introduction of a liquid sclerosant composition can produce an overpressure inside the foaming space, i.e. the (atmospheric) pressure inside the foaming space is higher than the ambient pressure.

A syringe may be used for aspiration or extraction of foam from the foaming space as well. The same syringe may then be used for injection into a patient's vein. The overpressure on the inside of the foaming space may provoke problems during aspiration, in that air or gas bubbles are present in the syringe. In order to avoid such problems, a pressure equalizer is provided which can decrease or avoid the overpressure in the foaming space.

In another aspect, the present disclosure provides a kit for the preparation of a foamed sclerosant composition comprising such a container and further comprising a syringe, optionally a syringe pre-filled with a liquid sclerosant composition.

In yet another aspect, a system is provided which comprises such a container and an actuator configured to be operatively coupled to the mixing element. The actuator may in particular be a magnetic stirrer.

In a further aspect, a method for preparing and extracting a foamed sclerosant composition is provided. The method comprises providing a syringe filled with a liquid sclerosant composition and providing a container as hereinbefore explained. The method furthermore comprises mating the syringe with the coupling and injecting the liquid sclerosant composition into the foaming space, operating the actuator to rotate the mixing element and create a foamed sclerosant composition, and extracting the obtained foamed sclerosant composition.

In a method according to this aspect, a substantially sterile and relatively easy method for preparing and extracting a foamed sclerosant composition is provided allowing the coupling of a syringe with the container having the foaming space.

In yet a further aspect, a foamed sclerosant composition obtainable by such a method is provided.

In some examples, the female coupling member or “socket” of the container body comprises a Luer taper. A female coupling member as used throughout the present disclosure may be regarded as a coupling member with a suitable receptacle or opening for receiving a male coupling member, and in the present case the male coupling member of a distal end of a syringe. The female coupling member may have the shape of a socket or a sleeve.

The Luer taper is a standardized system of small-scale fluid fittings used for making leak-free connections between a male-taper fitting and its mating female part on medical and laboratory instruments, including e.g. syringe tips.

The Luer taper of the female coupling member may be a female Luer slip. Alternatively, a Luer-lock may be used. Luer lock fittings are securely joined by means of a tabbed hub on the female fitting which screws into threads in a sleeve on the male fitting. Slip tip (Luer-slip) fittings simply conform to Luer taper dimensions and are pressed together and held by friction (they have no threads).

In some examples, the female coupling member for mating with a syringe further may comprise a one-way valve for the introduction of a liquid sclerosant composition. The one- way valve may ensure that the introduction of a liquid sclerosant composition can be done without compromising the sterile environment in the foaming space. The one-way valve may be a simple sheet or foil which is cantilever mounted to allow pivoting and thereby provide a one-way access to the foaming space. In some examples, the one-way valve may be arranged downstream from the point of injection of the liquid composition.

In some examples, the pressure equalizer may be a pressure release. The pressure release may be configured to release pressure only after a sclerosant foam has been created.

In some examples, the pressure equalizer or release may be manually activated. In some of these examples, the pressure equalizer or release comprises an orifice in the container body and a cover. The cover may be removable, releasable, or breakable. A user may simply remove the cover in order to reduce or avoid the differences in pressure in the inside of the foaming space, and a user can do so at a suitable moment. In some examples, instead of removing the cover, the cover may be broken. In other examples, the pressure equalizer or release may be an (automatic) release valve. “Automatic” herein means is that no manual activation by a user or healthcare professional is necessary.

In some examples, the mixing element configured to be operatively coupled with a rotatable actuator may be a disc comprising a magnetic part and a shape suitable for mixing. Such a mixing element can be activated and controlled by providing a changing magnetic field. In particular, the rotatable actuator may be configured to cause a rotating magnetic field, and optionally may be a magnetic stirrer.

In some examples, the gas may be a physiological gas, and optionally is a mixture of O2 and CO2. In other examples, the gas may be air. The foam prepared with physiological gases may be suitable for the treatment of certain patients and/or veins which cannot be treated by foam prepared with (sterile) air.

In some examples, the syringe used for the introduction of the liquid sclerosant composition remains mated with the female coupling member or “socket” while the foam is being created. If the syringe remains coupled with the container, the chances of contaminating the foam are reduced. After the foam has been created, the same syringe may be used for aspiration or extraction of the foam. Depending on how much foam is extracted, a lower pressure may be created inside the foaming space than outside the foaming space. The pressure equalizer may also be useful to again equalize the pressures so that a good quality foam may be extracted.

In some examples, the actuator may be operated at a first speed of rotation to create a foamed sclerosant composition, and the actuator is operated at a second speed of rotation while the foamed sclerosant composition is being extracted, wherein the first speed of rotation is different from the second speed of rotation. In particular, a first high speed of rotation may be used for rapid preparation of foam of good quality (e.g. small bubbles), and the rotation continues as the foam is being extracted. The continued rotation forces the created foam to the outer area of the foaming space, from where it can be aspirated.

In some examples, the foamed sclerosant composition obtainable by any of the methods or systems described herein may be for use in the treatment of varicose veins, spider veins or hemorrhoids.

### BRIEF DESCRIPTION OF THE DRAWINGS

Particular implementations of the present disclosure will be described in the following by way of non-limiting examples, with reference to the appended drawings, in which:

FIGS. 1A-1E schematically illustrate an example of a container for producing a foamed sclerosant composition;

FIGS. 2A-2F schematically illustrate an example of a mixing element which may be used in containers for preparing a foamed sclerosant composition; and

FIG. 3 schematically illustrates a sequence of steps in an example of a method for the production of a foamed sclerosant composition.

### DETAILED DESCRIPTION

The expression “therapeutically effective amount” as used herein, refers to the amount of the foamed composition that, when administered, is sufficient to treat the diseases to which it is addressed. The specific dose which depends on both the volume and the drug concentration of the foamed sclerosant composition to obtain a therapeutic benefit may vary depending on the particular circumstances of each patient.

As previously mentioned, an aspect of the present disclosure relates to a foamed sclerosant composition obtainable by any of the methods herein described. The expression “obtainable by” is used herein for defining the foamed sclerosant composition by its preparation process. In particular, it refers to the foamed composition that can be obtained through the preparation process which comprises the previously commented steps. For the purposes of the present disclosure, the expressions “obtainable”, “obtained” and similar equivalent expressions are used interchangeably and, in any case, the expression “obtainable” encompasses the expression “obtained”. Throughout the present disclosure, the terms sclerosant and sclerosing are used interchangeably. Similarly, sclerosing foam, and foamed sclerosant composition are used interchangeably as well.

For the purposes of the present disclosure, the term foamed sclerosing composition refers to a composition of a foam capable of bringing about a sclerosing effect, i.e. a composition for use as a medicament for intravenous injection, which is capable of causing an injury to the vessel wall by endothelial vacuolation of the epithelial cell membrane (the layer in contact with the bloodstream). Thus, the foamed sclerosing composition irritates the inner surface of the vein just producing the formed thrombus formation by platelets and aggregates. Similarly, the term liquid sclerosing composition refers to a composition in liquid form including a sclerosing agent. The liquid sclerosing composition forms an ingredient to obtain the foamed sclerosing composition. The sclerosing compositions according to examples of the present disclosure comprise a sclerosing agent and also a suitable vehicle which can be injected without toxicity in the affected veins. In some examples, the liquid is selected from sterile water (particularly distilled water) and physiological saline. Examples of sclerosing agents that can be present in the sclerosing compositions of examples of the present disclosure include, without limitation, polidocanol, sodium tetradecyl sulfate, chromated glycerin, hypertonic saline, sodium morrhuate and sclerodex (hypertonic saline in combination with dextrose). In a particular example, the sclerosing composition used comprises polidocanol and water. In another embodiment, the sclerosing composition further comprises glycerin.

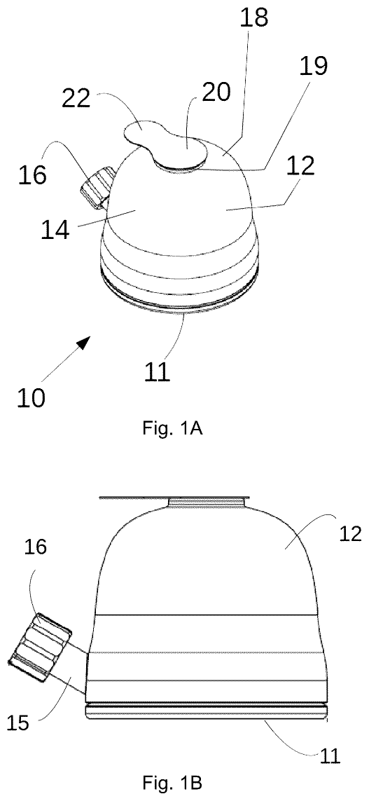

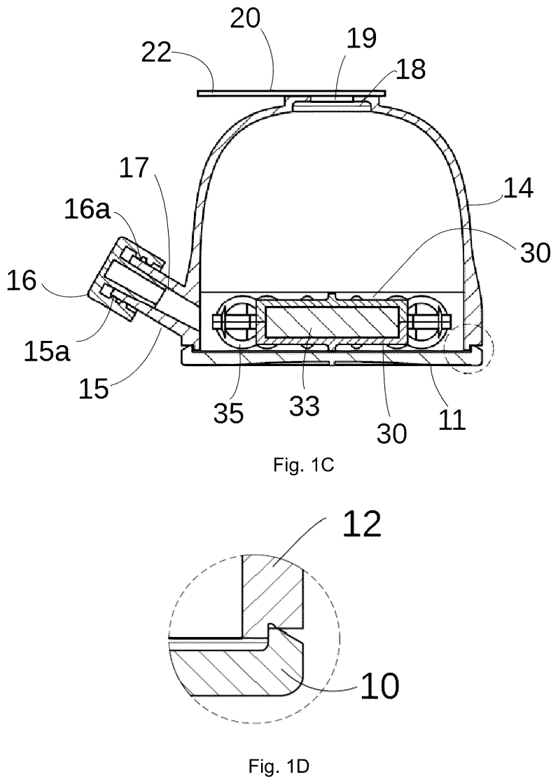

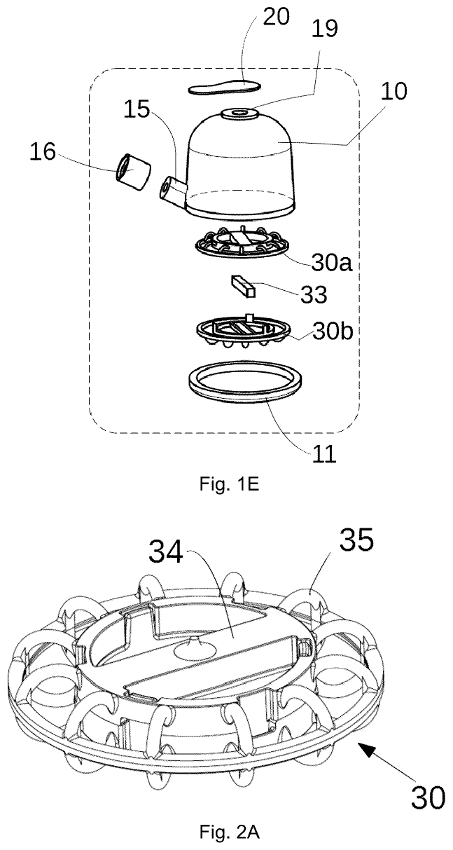

FIGS. 1A-1E schematically illustrate an example of a container for producing a foamed sclerosant composition. A container 10 according to this example may include a dome shaped container body 12 with a separately manufactured bottom 11. The dome shaped container body 12 may have a top 18 with an orifice 19. In this example, the orifice is closed by a removable cover 20.

The dome shape is formed by a curved side wall 14. In the interior of the container 10 a foaming space is defined. The container 10, both dome shaped body 12 and bottom 11, may be made from a suitable polymer such as Polyethylene terephthalate (PET) or other suitable materials such as plastics generally accepted in manufacture of medical devices.

In some examples, the material of which the container is made is transparent to allow visual inspection of the formation of the foam. The dome shaped container body 12 and the bottom part 11 may be made by injection molding within the same mold or by separate molds. Other manufacturing processes could also be used.

If manufactured separately, the bottom 11 and the dome shaped body 12 may be joined by ultrasonic welding or other appropriate ways for joining plastic components.

The container 10 according to this example may be sterilized and wrapped. For example, plastic foil or wrapper may be provided around the container as delivered to a medical professional.

A sterile gas may be introduced into the foaming space during manufacture and assembly. The gas may be air but may also be a mixture of physiological gases, e.g. a mixture of carbon dioxide (CO2) and oxygen (O2). As provided, the foaming space also includes a mixing element 30 (see FIG. 1C). The sterile container with previously introduced gas and mixing element may thus be provided to a medical professional.

In the shown example, the device of the present disclosure is sealed and closed, accesses to the interior of the container provided only by access ports, sockets and operational orifices that are located for best performance and reproducible production of improved foam. Restricted access to the interior of the device reduces risks of misuse and incorrect operation.

As shown in FIG. 1B, the container 10 according to this example further includes a female coupling member in the form of a socket 15 with a stopper or lid 16. The socket 15 may comprise external threads 15a and the stopper 16 may comprise mating threads 16a for easy attachment and detachment of the stopper. Alternative joints for socket and stopper may however also be foreseen, e.g. a press or snap fit.

The coupling socket 15 in this example comprises a Luer taper, and particularly a Luer slip. As shown in FIG. 1D, the Luer taper 17 may be sized such that it can mate with a tip of a syringe, in particular a hypodermic syringe. The tip of the syringe may be pressed into the Luer taper to provide a leak free fitting. In some examples, the syringe may be rotated to lock the syringe in the socket by means of a Luer lock.

In this example, the socket 15 is integrally formed with container 10 and in particular is provided on sidewall 14, in proximity to bottom 11. In some examples, the center of socket 15 is on the sidewall 14 up to 20 mm away from the bottom 11 of the container 10. Specifically, the center of socket 15 may be on the sidewall 14 up to 10 mm away from the bottom 11 of the container 10. However, other suitable positions for coupling socket 15 may be envisaged. The stopper 16 may be made from polyethylene or other suitable materials.

Although not shown in this specific example, the socket 15 or its connection to the foaming space may include a one-way valve, or any other element that facilitates passage from a liquid inside the syringe, and substantially impedes passage of gas from the inside to the outside.

In this example, the coupling socket 15 has an inclination with respect to a horizontal plane for easy insertion and extraction from the syringe.

Filling of the foaming space with a suitable sterile gas may take place through the same socket 15 that is used for injection of a sclerosant liquid composition, and potentially the same socket that is used for extracting the foam. An example of an assembly process is the following: after injection molding of the container 10, and before attachment of the bottom 11, the mixing element 30 is introduced into the foaming space of the container, and the housing is then closed. Sterile gas may be introduced through a suitable valve coupling which allows the introduction of the sterile gas (e.g. air, and/or a mixture of physiological gases) and evacuation of the gas previously present.

The container 10 may include a pressure equalizer, and in this specific example, a pressure release. In this specific example, an orifice 19 with a releasable cover 20 is provided as the pressure release. In other examples, the pressure release may be a relief valve, or an orifice with a breakable cover or tab. The function of the pressure release is to balance a pressure inside container 10 and a pressure outside the container. Equilibration of pressures can avoid potential formation of undesirable large bubbles in the sclerosant foam.

Orifice 19 may have a relatively small diameter, sufficient for gas exchange. Without limitation, orifice 19 can be 0.5 to 5 mm in diameter. Specifically, orifice 19 may be between 1 and 3 mm in diameter.

Orifice 19 may particularly be located where it is not exposed to the foam created, for example at a high point of container 10. In this example, orifice 19 is covered by a releasable cover 20 such as an aluminum foil sealed over orifice 19. As may be seen for example in FIGS. 1B and 1C, the cover 20 may have a shape adapted for easily pulling off the cover and may include a handle or grip 22 which a user may grip for handling. The grip may be provided at one end of the cover 20, whereas the opposite end may seal the orifice 19. Orifice 19 may also include a filter element or microbial barrier to reduce potential exposure of the foam to airborne particles and microbes upon equilibration of pressures and withdrawal of the foam.

Opening of orifice 19 may be carried out at different moments in the operation of the device. In a specific example disclosed herein orifice 19 is maintained closed in operation during initial formation of the foam and is only exposed shortly before withdrawal of the foam contained in container 10. Orifice 19, or in general the pressure equalizer or release, may be configured such that it only becomes operational immediately prior or simultaneously to the withdrawal of the foam.

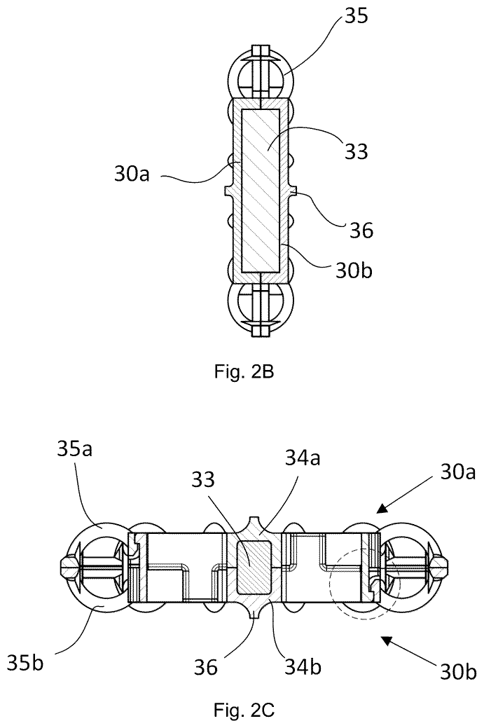

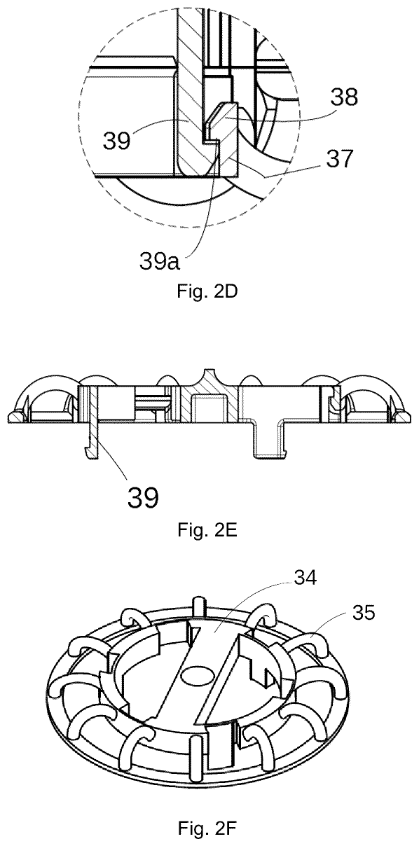

Details of mixing element 30 may particularly be seen in FIGS. 2A-2F. The mixing element 30 in this example may be a disc and, as may be seen e.g. in FIG. 2B, it may comprise a magnetic element 33, in particular a magnetic core. As shown e.g. in FIG. 2A, the magnetic element may be provided in a receptacle 34, e.g. a centrally arranged receptacle 34. The magnetic element 33 may be a permanent magnet or may be made from a (soft) ferromagnetic material. When using a (soft) ferromagnetic material, rather than a permanent magnet, the element 33 obtains its magnetic properties because of its arrangement in the presence of a magnet, e.g. the magnet of a magnetic stirrer. The magnetic properties then disappear when the magnet is removed. The magnetic element 33 may be made e.g. from neodymium, or other materials with good magnetic properties. In another example, the magnetic element may be made of stainless steel.

Around an outer perimeter of the disc a plurality of suitably shaped features for mixing may be provided. Such features may include e.g. blades, rings or teeth, which ensure mixing a liquid and gas by creating sufficient turbulence and mixing of the various components as the disc is rotated. Mixing may be increased by forced passage through openings or spaces in the disc.

The disc may be freely arranged, i.e. not supported or suspended by any element on the sidewall or the top of the container. The mixing element 30 may comprise a central protrusion 36 which is configured to reduce contact and friction between a bottom of the mixing element with the bottom of the container. The protrusion 36 may be tapered and pointed. When the mixing element starts to rotate, the protrusion 36 maintains contact with the bottom of the container body.

In this particular example, ring shaped mixing features 35 are shown, but it should be clear that many other shapes may be suitable.

In this particular example, as shown e.g. in FIG. 2C, the disc may be made by assembly of an upper half 30A and lower half 30B. Each of the halves 30A, 30B may include half rings 35A, 35B, half compartments 34A, 34B and suitable coupling features. Half compartment 34A, once coupled with half compartment 34B forms a receptacle for the magnetic element 33.

Each of the halves may be made from a suitable polymer, such as polyethylene and may be made e.g. by injection molding. As may be seen e.g. in FIG. 2D, the halves may include suitable joining features, e.g. fingers 37 with bulging tips 38 which can snap fit over corresponding bulges 39a on a sidewall 39 of the other half.

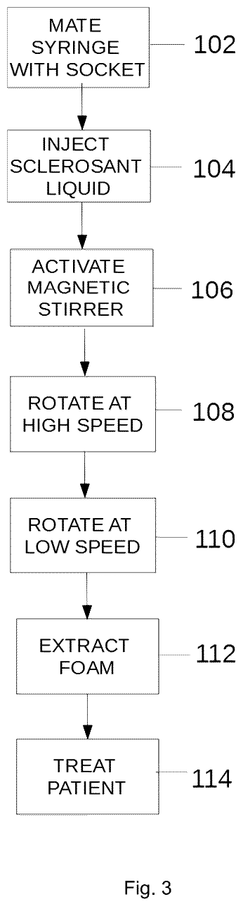

One example of preparing of a sclerosant foam with a container 10 and mixing element 30 may be illustrated with reference to FIG. 3.

A container according to the previously shown example or similar may be provided. A syringe filled with a sclerosant liquid composition may be provided as well. In an example, such a syringe may be provided as a pre-filled syringe, possibly as part of a kit also comprising container 10. Alternatively, the syringe may be filled with the sclerosant liquid immediately prior to the preparation of the foam.

In one particular example, the sclerosing composition may comprise a solution of a sclerosing agent, such as e.g. polidocanol, in a liquid, such as water or physiological saline, at a concentration from 2 mg to 30 mg in 1 mL liquid (which corresponds to 0.20-3.0%) (w/v). In another example, the sclerosing composition may comprise a solution of a sclerosing agent, such as polidocanol, in a liquid, such as water or physiological saline, at a concentration from 2 mg to 5 mg in 1 mL liquid (which corresponds to 0.20-0.50% (w/v)). With the devices and methods described herein, it has been found that even at very low concentrations e.g. 0.2% or 0.3% (w/v), still a stable foam may be obtained, contrary to e.g. Tessari's method.

In another particular example, the sclerosing composition may comprise a solution of polidocanol in water or physiological saline at a concentration of 5 mg/mL.

In another particular example, the sclerosing composition may comprise a solution of polidocanol in water or physiological saline at a concentration of 20 mg/mL.

In another particular example, the liquid composition may include Sodium tetradecyl sulfate (STS) at a concentration of 0.2-3%.

The volume of the syringe may be e.g. 5 cc, 10 cc or 20 cc. The syringe may comprise e.g. 2 ml or more of liquid sclerosant composition.

At block 102, the syringe may be mated with socket 15 by insertion of the syringe. Prior to this, the stopper 16 is removed from the socket 15. Immediately after removing stopper 16, the syringe may be introduced to avoid any contamination. In further examples, the coupling socket or container body may include a one-way valve or a selectively displaceable element to avoid any contamination. In yet further examples, the container may be provided with a slight overpressure (i.e. higher than ambient pressure) to avoid potential contamination once stopper 16 is removed. Combinations thereof are also possible. In examples, the stopper 16 may be configured for single use only. I.e. the stopper 16 may be in the form of a lid or seal that is broken, damaged or otherwise made unusable when removed.

Once a leak free fitting between the syringe and the socket 15 is established, the liquid composition inside the syringe may be injected at block 104 and can enter the foaming space. Because liquid is introduced into a closed system a slight overpressure can be created inside the foaming space, that is, the pressure inside the foaming space is higher than the external atmospheric pressure. Any air or gas inside the syringe may also be injected into the foaming space, although this may be done at any moment prior to extraction of the foam.

The previously described actions may be carried out in particular after placement of container 10 on a magnetic stirrer. This may be a standard magnetic stirrer which may typically be found in many laboratories. In some other examples, the magnetic stirrer may be specifically adapted to the container and include a receptacle for receiving the container. A slight press fit between the container and the receptacle may be provided.

Then, at block 106, the magnetic stirrer may be activated. The mixing element may thus be set in rotation. The mixing element, e.g. the disc with magnetic element that was previously described may rotate on the bottom of the container. Depending on the type of foam to be created, depending on the liquid sclerosant composition, and depending on the gas in the foaming space, different revolution speeds and times may be used. In some examples, the magnetic stirrer may include a memory with a number of predefined programs suitable for different foams, such that a user only needs to select a suitable program.

It is noted that it can be advantageous not to remove the syringe during the formation of the foam and maintain it coupled to the container.

In particular examples, the magnetic stirrer is first set to a high first speed at block 108, which is reached in a short time, for example a few seconds. Such a high first speed may be between 2,000 and 5,000 RPM, and particularly between 2,500 and 4,000 RPM. After as little as 15 seconds, foam may be created.

In order to increase the stability and quality of the foam, the high speed of rotation may be maintained however. After a first time period, which may be 30-90 seconds, particularly around 60 seconds, the speed of the magnetic stirrer may be reduced to a second speed at block 110.

The second speed may be chosen such that the foam that has been created is pushed to the outer perimeter of the foaming space due to centrifugal forces. The second speed may be lower than 1,500 RPM. In an example, the second speed may be between 200 and 1,200 RPM, specifically around 1,000 RPM.

Before extraction of foam at block 112, the releasable cover 20 may be removed from the pressure release orifice 19 to equilibrate the overpressure inside with the outside atmospheric pressure. Preferably, orifice 19 is maintained open during extraction of the sclerosant foam from the container to avoid creation of an underpressure inside the foaming space and syringe during the extraction. Equilibration of pressures is directed towards avoiding potential formation of undesirable large bubbles in the sclerosant foam within the collection syringe.

After extraction of the sclerosant foam, the foam may be used in the treatment of a patient at block 114.

The volume of container 10 as disclosed herein may be, for example, 30 ml. Such a container was used in various experiments. With methods according to the examples disclosed herein, approximately 10 ml of stable foam was obtained and easily extracted when introducing 2 ml of liquid sclerosant composition (of any of the hereinbefore mentioned concentrations).

When introducing 4 ml of liquid sclerosant composition, about 20 ml of stable foam was obtained and easily extracted.

For reasons of completeness, various aspects of the present disclosure are set out in the following numbered clauses:

Clause 1. A container for the production of a foamed sclerosant composition, the container comprising:
a sealed sterile container body for foaming comprising one or more sidewalls extending between a top and a bottom of the container body, a foaming space being defined in an interior of the container body,
the foaming space containing a sterile gas and a mixing element configured to be operatively coupled with a rotatable actuator without the actuator entering into the foaming space, wherein
the container comprises a female coupling member for mating with a syringe, and
a pressure equalizer for equalizing a pressure inside the foaming space and outside the foaming space.

Clause 2. A container according to clause 1, wherein the female coupling member comprises a Luer taper.

Clause 3. A container according to clause 2, wherein the Luer taper is a female Luer slip.

Clause 4. A container according to any of clauses 1-3, wherein the female coupling member comprises a lid for closing the coupling.

Clause 5. A container according to clause 4, wherein the lid optionally has threads for mating with threads on the coupling.

Clause 6. A container according to any of clauses 1-5, wherein the female coupling member for mating with a syringe further comprises a one-way valve for the introduction of a liquid sclerosant composition.

Clause 7. A container according to any of clauses 1-6, wherein the female coupling member is located in the sidewall of the container body.

Clause 8. A container according to clause 7, wherein the female coupling member is arranged at or near an edge of the bottom surface of the container body, and optionally has inclination with respect to a horizontal plane.

Clause 9. A container according to any of clauses 1-8, wherein the pressure equalizer is a pressure release for releasing an overpressure inside the foaming space, and optionally wherein the pressure release is configured to release pressure only after a sclerosant foam has been created.

Clause 10. A container according to any of clauses 1-9, wherein the pressure release comprises an orifice in the container body and a releasable cover.

Clause 11. A container according to clause 10, wherein the orifice is located at the top of the container body.

Clause 12. A container according to clause 10 or 11, wherein the releasable cover comprises a grip for pulling off the releasable cover.

Clause 13. A container according to any of clauses 10-12, wherein the releasable cover is heat sealed over the orifice.

Clause 14. A container according to any of clauses 1-13, wherein the mixing element configured to be operatively coupled with a rotatable actuator is a disc comprising a magnetic part and a shape suitable for mixing, and wherein the rotatable actuator is configured to cause a rotating magnetic field, and optionally is a magnetic stirrer.

Clause 15. A container according to clause 14, wherein the mixing element is a disc comprising a magnetic part with a plurality of blades around its circumference.

Clause 16. A container according to clause 15, wherein the blades include on or more of the following: rings, vertical teeth and horizontal teeth.

Clause 17. A container according to any of clauses 15-16, wherein the disc comprises an upper disc and a lower disc, configured to be coupled to each other, the upper and lower disc defining a compartment for housing the magnetic part.

Clause 18. A container according to clause 17, wherein the upper and the lower disc are configured to be snap fitted together.

Clause 19. A container according to any of clauses 15-18, wherein the disc comprises a central protrusion configured to enter into contact with the bottom of the container body.

Clause 20. A container according to any of clauses 1-19, wherein the gas is a physiological gas, and optionally is a mixture of 02 and CO2.

Clause 21. A container according clause 20, wherein a percentage of 02 is between 70%-50%, and the remainder is CO2.

Clause 22. A container according to any of clauses 1-21, enclosed in a sterile packaging.

Clause 23. A kit for the preparation of a foamed sclerosant composition comprising a container according to any of clauses 1-22, further comprising a syringe, optionally a syringe pre-filled with a liquid sclerosant composition.

Clause 24. A kit for the preparation of a foamed sclerosant composition according to clause 23, wherein the syringe is a Luer lock syringe comprising one of the following volumes: 2.5 cm3, 5 cm3, 10 cm3 or 20 cm3.

Clause 25. A kit for the preparation of a foamed sclerosant composition according to any of clauses 23-24, wherein the syringe comprises 2-4 ml of a liquid sclerosant composition.

Clause 26. A kit according to any of clauses 23-25, wherein the volume ratio gas: liquid in the foaming space once the liquid sclerosant composition is introduced into the container body is between 7.5:1 and 20:1.

Clause 27. A kit for the preparation of a foamed sclerosant composition according to any of clauses 23-26, wherein the liquid sclerosant composition is a solution of a drug in distilled water or a saline.

Clause 28. A system for the production of a foamed sclerosant composition comprising a sterile container according to any of clauses 1-22 and an actuator configured to be operatively coupled to the mixing element.

Clause 29. A system for the production of a foamed sclerosant composition according to clause 28, wherein the actuator is a magnetic stirrer.

Clause 30. A method for preparing and extracting a foamed sclerosant composition comprising:
providing a syringe filled with a liquid sclerosant composition;
providing a container, and optionally a container according to any of clauses 1-22;
   mating the syringe with the coupling and injecting the liquid sclerosant composition into the foaming space;
operating the actuator to rotate the mixing element and create a foamed sclerosant composition, extracting the obtained foamed sclerosant composition.

Clause 31. A method according to clause 30, wherein the syringe remains mated with the coupling while the foam is being created.

Clause 32. A method according to clause 30, wherein the obtained foamed sclerosant composition is extracted by aspirating the syringe.

Clause 33. A method according to any of clauses 30-32 wherein the actuator is operated to rotate the mixing element at a first speed of rotation of 5,000 RPM or below, and preferably rotating the actuator to rotate the mixing element at a first speed of rotation of between 2,000 and 4,500 RPM during a first period of time.

Clause 34. A method according to clause 33, comprising rotating the actuator to rotate the mixing element for a first period of time at a first speed of rotation with a varying speed.

Clause 35. A method according to clause 33 or 34, wherein first period of time is between 15 and 90 seconds, specifically between 30 and 75 seconds, more specifically between 30 and 60 seconds.

Clause 36. A method according to any of clauses 30-35, wherein the actuator is operating while the obtained foamed sclerosant composition is being extracted.

Clause 37. A method according to any of clauses 30-36, wherein the actuator is operated at a first speed of rotation to create a foamed sclerosant composition, and the actuator is operated at a second speed of rotation while the foamed sclerosant composition is being extracted, wherein
the first speed of rotation is different from the second speed of rotation.

Clause 38. A method according to clause 37, wherein the second speed of rotation is lower than the first speed of rotation.

Clause 39. A method according to clause 37 or 38, comprising rotating the actuator to rotate the mixing element at a second speed of rotation of 1,500 RPM or below, specifically between 500-1,200 RPM, more specifically around 1,000 RPM.

Clause 40. A method according to any of clauses 30-39, further comprising before extracting the obtained foamed sclerosant composition, releasing an overpressure inside the foaming space.

Clause 41. A method according to any of clauses 30-40, wherein the liquid sclerosant composition is a solution of the sclerosant drug in water, optionally distilled water, or a saline.

Clause 42. A method according to clause 41, wherein a concentration of the drug is 0.20-3.0% (w/v).

Clause 43. A method according to clause 42, wherein the concentration of the drug is 0.20-0.50% (w/v).

Clause 44. A foamed sclerosant composition obtainable by a method according to any of clauses 30-43.

Clause 45. A foamed sclerosant foam according to clause 44, wherein the ratio of liquid: gas in the sclerosant foam is between 1:4 and 1:7.

Clause 46. A foamed sclerosant composition according to clause 44 or 45, for use in the treatment of varicose veins.

Clause 47. A foamed sclerosant composition according to clause 44 or 45, for use in the treatment of spider veins.

Clause 48. A foamed sclerosant composition according to clause 44 or 45, for use in the treatment of hemorrhoids.

Clause 49. A magnetic stirrer having one or more magnets for creating a rotating magnetic field, wherein the magnetic stirrer has a control, the control including a processor and memory
wherein the memory includes instructions corresponding to one or more predetermined rotation programs, such that when the instructions are executed by the processor, the magnets create a rotating magnetic field according to the predetermined rotation programs, the predetermined rotation programs corresponding to rotations defined in any of clauses 33-39.

Clause 50. The magnetic stirrer according to clause 49, wherein the one or more magnets are rotating magnets.

Clause 51. The magnetic stirrer according to clause 49, wherein the one or more magnets are static electromagnets.

Clause 52. The magnetic stirrer according to any of clauses 49-51, further comprising a user interface, wherein the user interface enables selection by a user of the predetermined rotation programs.

Clause 53. The magnetic stirrer according to clause 52, wherein the user interface includes a touch screen display.

Clause 54. The magnetic stirrer according to clause 52 or 53, wherein the predetermined rotation programs are identified by one or more of the following: the gas in the foaming space, and the liquid sclerosant composition, specifically a concentration of the drug in the liquid sclerosant composition.

Although only a number of examples have been disclosed herein, other alternatives, modifications, uses and/or equivalents thereof are possible. Furthermore, all possible combinations of the described examples are also covered.

1. A method for preparing and extracting a foamed sclerosant composition comprising:
providing a container having:
a sealed container body for foaming comprising one or more sidewalls extending between a top and a bottom of the container body, a foaming space being defined in an interior of the container body, the foaming space containing a sterile gas and a mixing element configured to be operatively coupled with a rotatable actuator without the actuator entering into the foaming space, the interior of the container body being sterile, the container further including a female coupling member for mating with a syringe, and a pressure equalizer for equalizing a pressure inside the foaming space and a pressure outside the foaming space;
the method further comprising:
providing a syringe filled with a liquid sclerosant composition;
mating the syringe with the female coupling member and injecting the liquid sclerosant composition into the foaming space through the female coupling member;
operating the actuator to rotate the mixing element to create a foamed sclerosant composition in the foaming space; and
extracting the obtained foamed sclerosant composition from the foaming space.

2. The method according to claim 1, further comprising maintaining the syringe mated with the female coupling member while the foam is being created.

3. The method according to claim 2, further comprising extracting the obtained foamed sclerosant composition from the foaming space by aspirating the syringe while the syringe is maintained mated with the female coupling member.

4. The method according to claim 1, wherein the actuator is operated to rotate the mixing element at a first speed of rotation of between 2,000 and 5,000 RPM during a first period of time.

5. A method according to claim 4, wherein the first period of time is between 15 and 90 seconds.

6. The method according to claim 4, wherein the actuator is operated at a second speed of rotation less than the first speed of rotation while the obtained foamed sclerosant composition is extracted.

7. The method according to claim 6, wherein the second speed of rotation is between 200 to 1,200 RPM.

8. The method according to claim 1, further comprising lowering a pressure inside the foaming space before extracting the obtained foamed sclerosant composition from the foaming space.
